(19) United States
(12) Patent Application Publication
(10) Pub. No.: US 2009/0182387 A1
(43) Pub. Date: Jul. 16, 2009

(54) Energy-enhanced health-promoting glove structure

(76) Inventor: Hung-Tao Liu, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/222,315

(22) Filed: Aug. 7, 2008

(30) Foreign Application Priority Data
Jan. 16, 2008 (TW) 097201029

(51) Int. Cl. A61N 1/39; A41D 19/00

(52) U.S. Cl. 607/1; 2/159

(57) ABSTRACT

An energy-enhanced health-promoting glove structure is disclosed, wherein a glove body is provided with an energy-generating material that is distributed on at least one surface of the glove body at different densities and over different extents in accordance with a distribution density of acupuncture points on a human hand and requirements in selecting the acupuncture points on the hand, so that stimulating and regulating effects of various strengths can be produced at different meridians and acupuncture points of the hand. In addition, the glove body remains perfectly soft in areas that need not produce such effects and therefore provides a more comfortable wearing experience.

### BACKGROUND OF THE INVENTION

1. Technical Field

The present invention relates to a glove structure, and more particularly, to an energy-enhanced health-promoting glove structure, wherein a glove body is provided with an energy-generating material that is distributed on at least one surface of the glove body at different densities and over different extents in accordance with a distribution density of acupuncture points on a human hand and requirements in selecting the acupuncture points on the hand, so that stimulating and regulating effects of various strengths can be produced at different meridians and acupuncture points of the hand. In addition, the glove body remains perfectly soft in areas that need not produce such effects and therefore provides a more comfortable wearing experience.

2. Description of Related Art

People in a modern society live a busy life. Stressful lifestyles and lack of proper exercise and relaxation lead to a variety of diseases of civilization. Therefore, numerous kinds of health-promoting or massage devices for rapidly relieving fatigue, loosing up strained muscles or improving the circulation of qi and blood have been developed and put on the market. Of all these devices, many are designed to produce certain effects on the human hand and foot. This is mainly because therapy based on the acupuncture points on the hand and foot has been proven effective by traditional Chinese medicine. More specifically, the human hand and foot have a lot of acupuncture points distributed thereon that correspond to and reflect different parts or organs of the human body. In addition, some of the major meridians run through the hands and feet. Therefore, by massaging, acupuncturing or performing magnetic therapy, medicated patches, herbal fuimigation and washing on the acupuncture points and reflective zones on the hands and feet, the entire body or individual systems thereof can be cured of diseases or regulated so as to recuperate or maintain good health. According to traditional Chinese medicine, this kind of treatment falls into the category where internal disorders are dealt with by external means. Besides, as the old Chinese sayings go, “the rich take medicine, the poor scald their feet” and “the rich eat ginseng, the poor knead their soles”. It is also widely accepted in the Chinese medical community that “aging begins with the feet”, “a pair of ruddy feet indicate both physical and mental health” and “the three Yin meridians in the hand originate from the chest while the three Yang meridians in the hand go to the head”. This can be explained by the fact that a great number of nerve endings are distributed in the human feet and hands, where qi and blood circulate relatively slowly, and that the functioning of internal organs depends on the circulation of qi and blood, or rather, as ancient Chinese put it, “qi is the master of blood while blood is the mother of qi”, meaning qi and blood are dependent upon and contributive to each other. Therefore, illnesses may take place wherever the circulation is congested. Particularly, the hand, where the hand meridians pass through, is the confluence of qi and blood among the three Yin and three Yang meridians of the hand. More specifically, the hand is where the three Yang meridians of the hand terminate and the three Yin meridians of the hand begin. These three Yin and three Yang meridians of the hand are further connected with other meridians in the human body. Therefore, by sufficiently energizing the three Yin and three Yang meridians of the hand, all the other meridians of the human body can be directly or indirectly invigorated, so that diseases can be cured and the body strengthened. Hence, massaging the acupuncture points on the hand, as practiced in traditional Chinese medicine, is no other than a way to accelerate blood circulation, improve nutrient transport in the body and promote health.

Among the aforesaid health-promoting or massage devices are articles of clothing, including clothes, gloves, socks, shoes, head coverings and so forth, wherein gloves work on the hands. Nowadays, the most common design is to apply and couple an energy-generating element, in the form of particles, to an inner surface or inner layer of a glove. Although the final product can be conveniently worn, the particles may make a wearer uncomfortable if they are not distributed uniformly over the entire product. On the other hand, an overly dense distribution of the particles may reduce the overall softness of the product and restrict the design and application thereof. An alternative design is to impregnate fibers or fabrics with the energy-generating element, which are then made into the desired objects. In this case, however, the production cost is higher and, in order to satisfy users' need of a comfortable feel on the body, the materials used to make the objects themselves tend to limit the application, and impair the strength, of the final products, thereby leaving much room for improvement. Moreover, the foregoing two designs are suitable for comprehensive application of the energy-generating element, and yet the acupuncture points on the hand are not distributed uniformly over the entire hand. In other words, the acupuncture points are abundant in some areas, but sparse in other areas, of the hand. Therefore, according to the requirements of traditional Chinese medicine in meridian circulation and the replenishment and purgation of acupuncture points or the theory of qi and blood conduction, if the energy-generating element is applied comprehensively and uniformly, the effect may be compromised, so that improvement is imperative in this respect.

### BRIEF SUMMARY OF THE INVENTION

A primary objective of the present invention is to design an energy-enhanced health-promoting glove structure which can be more comfortably worn and is configured to better conform to the physical therapy principles of traditional Chinese medicine. More particularly, a glove body is provided with an energy-generating material (comprising a negative electromagnetic energy-generating material, a positive electromagnetic energy-generating material, or a mineral having energy), wherein the energy-generating material is distributed over a large extent and/or in a great quantity on parts of the glove body corresponding in position to areas of the hand where acupuncture points are densely distributed, and over a small extent and/or in a small quantity on parts of the glove body corresponding in position to areas of the hand where acupuncture points are sparsely distributed. Thus, energy level effects of sufficient but varied strengths can be generated in different areas of the hand to facilitate the conduction of qi and blood, increase the momentum for qi and blood transportation, and thereby improve the circulation of qi and blood.

Another objective of the present invention is to apply an energy-generating material to an article of clothing only in areas corresponding in position to acupuncture points that need to be acted upon, whereas other areas of the article of clothing corresponding in position to acupuncture point that need not be acted upon are provided with a reduced quantity of, or not provided at all with, the energy-generating material, thereby satisfying the requirements of physical therapy in traditional Chinese medicine, overcoming the problem that the article of clothing might have a less pleasing texture due to the application of the energy-generating material, and reducing the overall production cost.

A further objective of the present invention is to apply an energy-generating material, in the form of a coating material or an adhesive material, to a surface of a glove body at a very small thickness, so that the applied area is allowed to remain sufficiently soft and will not press too hard on or even irritate a user's skin when the glove body is worn, thereby giving the user a more comfortable wearing experience.

### BRIEF DESCRIPTION OF THE SEVERAL VIEWS OF THE DRAWINGS

The physical structure and effects of the present invention can be best understood by referring to the following detailed description of the preferred embodiment and the accompanying drawings, wherein:

FIG. 1 is a schematic drawing showing acupuncture points on a human palm;

FIG. 2 is a schematic drawing showing acupuncture points on the back of a human hand;

FIG. 3 is a schematic structural drawing of the present invention, showing a side thereof corresponding to the palm;

FIG. 4 is a schematic drawing of the present invention worn on a user's hand, showing the side corresponding to the palm;

FIG. 5 is a schematic structural drawing of the present invention, showing a side thereof corresponding to the back of the hand;

FIG. 6 is a schematic drawing of the present invention worn on a user's hand, showing the side corresponding to the back of the hand;

FIG. 7 is a schematic drawing of the present invention worn on a user's hand, showing qi and blood conduction by the present invention in a purging state; and

FIG. 8 is a schematic drawing of the present invention worn on a user's hand, showing qi and blood conduction by the present invention in a replenishing state.

### DETAILED DESCRIPTION OF THE INVENTION

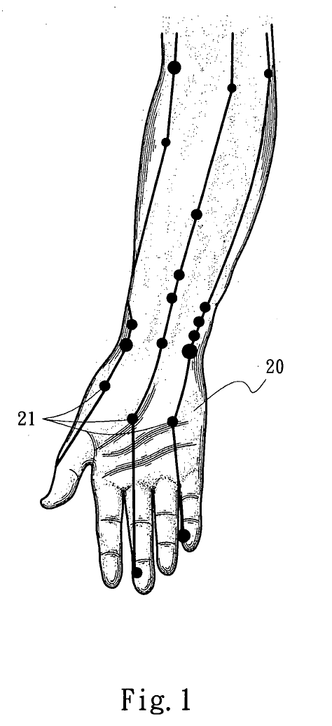

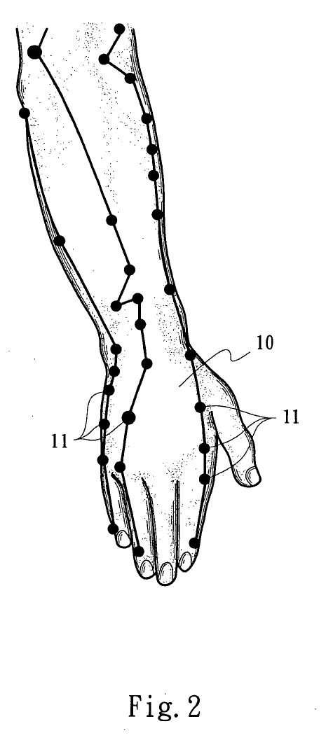

FIGS. 1 and 2 illustrate distributions of acupuncture points 11 and 21 (shown as black dots) on the back 10 and the palm 20 of a human hand, respectively. As mentioned earlier, the acupuncture points are distributed more densely in certain areas than in others. By comparing FIG. 1 with FIG. 2, which show the distributions of acupuncture points on the palm 20 and the back 10 of the hand, respectively, it can be found that the acupuncture points 21 on the palm 20 are distributed less densely than the acupuncture points 11 on the back 10 of the hand.

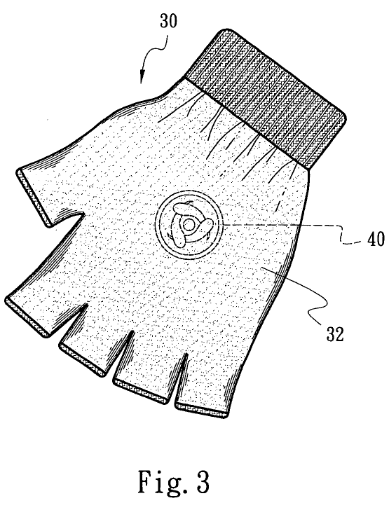

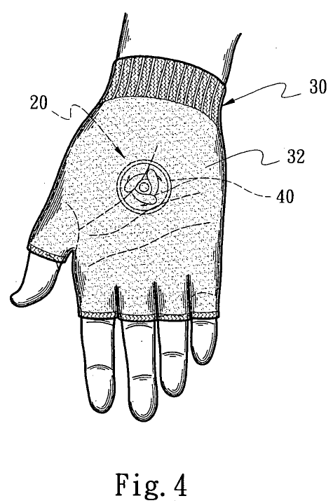

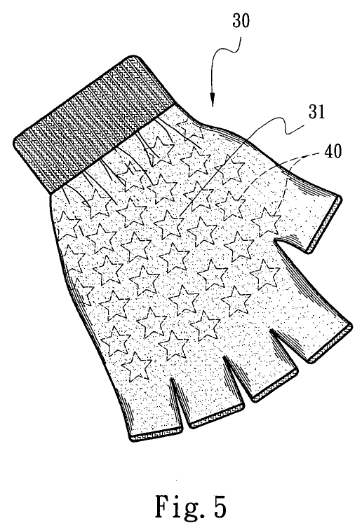

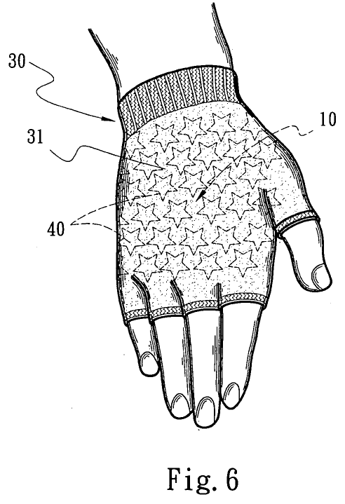

Referring to FIGS. 3 to 6, the present invention comprises a glove body 30, wherein an energy-generating material 40 (including a negative electromagnetic energy-generating material, a positive electromagnetic energy-generating material, or a mineral or mineral powder having energy) is printed, coated or adhered on at least one (inner) surface of the glove body 30. The extent to which and the quantity in which the energy-generating material 40 is distributed on the glove body 30 depend on and correspond to the extent to which and the density at which the acupuncture points are distributed on a wearer's hand. For example, a part 31 of the glove body 30 corresponding in position to the back 10 of the hand is provided densely with a large quantity of the energy-generating material 40, so as to produce relatively intense effects on the relatively dense, abundant acupuncture points in that portion of the hand (such as the acupuncture points of Zhongzhu, Yemen, Houxi, Qiangu, Hegu, Sanjian and Erjian). On the other hand, a part 32 of the glove body 30 corresponding in position to the palm 20, where a small number of acupuncture points 21 (such as the acupuncture points of Laogung, Shaofu and Yuji on a central portion of the palm) are sparsely distributed, is either provided with a single large spot of the energy-generating material 40 or provided sparsely with a small quantity of the energy-generating material 40.

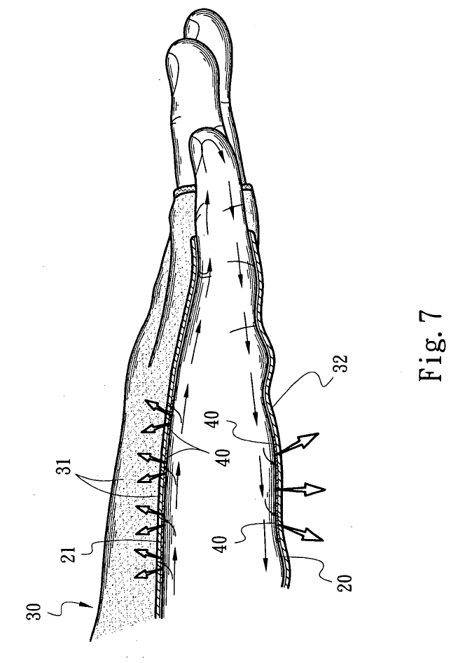

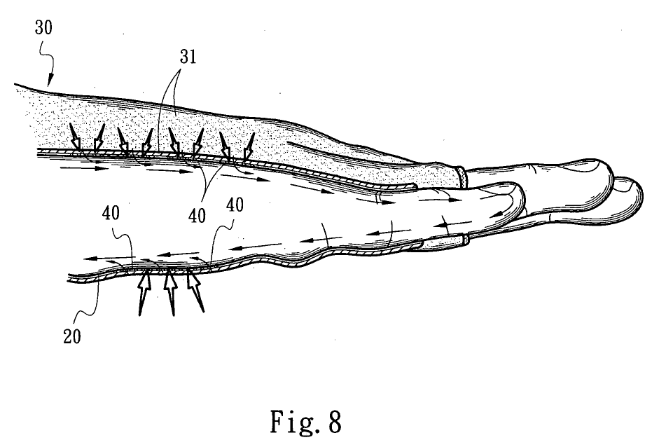

Referring now to FIGS. 7 and 8, according to the glove structure described above, the different quantities of the energy-generating material 40 distributed at varied densities on the glove body 30 can generate different energy levels in different areas of the hand where the acupuncture points 11 and 21 are located, thereby improving the circulation of qi and blood among the acupuncture points 11 and 21, and producing various levels of conducting, replenishing and purging effects on the acupuncture points 11 and 21. Hence, an auxiliary force is generated to energize the meridians along which qi and blood are circulated, so as to enhance metabolism.

Furthermore, since the energy-generating material 40 of the present invention is coated, printed or adhered at a very small thickness on parts of the glove body 30 corresponding in position to the acupuncture points on the hand, rather than applied to the entire glove body 30 in the form of particles having a considerable thickness, the glove body 30 not only remains sufficiently soft in those parts where the energy-generating material 40 is applied, but also preserves the softness and texture of the material of the glove body 30 in areas where energy effects are not required. As the unfavorable effects of the energy-generating material 40 on how the glove body 30 may feel on the wearer's hand are reduced, the glove body 30 can be more comfortably worn.

In conclusion, the present invention provides an energy-enhanced health-promoting glove structure that better conforms to the principles of physical therapy and health care in traditional Chinese medicine, wherein the glove structure is further designed to provide a more comfortable wearing experience. Therefore, undoubtedly, a health-promoting glove of the present invention involves an inventive step.

1. An energy-enhanced health-promoting glove structure, comprising a glove body having a surface at least partially provided with an energy-generating material, wherein the energy-generating material is distributed densely or sparsely, depending on a density and a number of acupuncture points distributed on a human hand.

2. The energy-enhanced health-promoting glove structure of claim 1, wherein the energy-generating material is densely distributed in the form of a large number of spots on a part of the glove body corresponding in position to an area of the human hand where the acupuncture points are densely distributed, and the energy-generating material is sparsely distributed or is disposed in the form of a single large spot on a part of the glove body corresponding in position to an area of the human hand where the acupuncture points are sparsely distributed.

3. The energy-enhanced health-promoting glove structure of claim 1, wherein the energy-generating material is distributed, at a high density and over a large extent, on a part of the glove body corresponding in position to a back of the human hand.

4. The energy-enhanced health-promoting glove structure of claim 2, wherein the energy-generating material is distributed, at a high density and over a large extent, on a part of the glove body corresponding in position to a back of the human hand.

5. The energy-enhanced health-promoting glove structure of claim 1, wherein the energy-generating material is distributed at a lower density on a part of the glove body corresponding in position to a palm of the human hand.

6. The energy-enhanced health-promoting glove structure of claim 2, wherein the energy-generating material is distributed at a lower density on a part of the glove body corresponding in position to a palm of the human hand.

7. The energy-enhanced health-promoting glove structure of claim 5, wherein the energy-generating material is provided only on a part of the glove body corresponding in position to a central portion of the palm.

8. The energy-enhanced health-promoting glove structure of claim 1, wherein the energy-generating material is coated, printed or adhered on the glove body at a very small thickness.

9. The energy-enhanced health-promoting glove structure of claim 2, wherein the energy-generating material is coated, printed or adhered on the glove body at a very small thickness.

10. The energy-enhanced health-promoting glove structure of claim 3, wherein the energy-generating material is coated, printed or adhered on the glove body at a very small thickness.

11. The energy-enhanced health-promoting glove structure of claim 5, wherein the energy-generating material is coated, printed or adhered on the glove body at a very small thickness.

12. The energy-enhanced health-promoting glove structure of claim 7, wherein the energy-generating material is coated, printed or adhered on the glove body at a very small thickness.

13. The energy-enhanced health-promoting glove structure of claim 1, wherein the energy-generating material comprises a negative electromagnetic energy-generating material.

14. The energy-enhanced health-promoting glove structure of claim 2, wherein the energy-generating material comprises a negative electromagnetic energy-generating material.

15. The energy-enhanced health-promoting glove structure of claim 3, wherein the energy-generating material comprises a negative electromagnetic energy-generating material.

16. The energy-enhanced health-promoting glove structure of claim 5, wherein the energy-generating material comprises a negative electromagnetic energy-generating material.

17. The energy-enhanced health-promoting glove structure of claim 8, wherein the energy-generating material comprises a negative electromagnetic energy-generating material.

18. The energy-enhanced health-promoting glove structure of claim 1, wherein the energy-generating material comprises a positive electromagnetic energy-generating material.

19. The energy-enhanced health-promoting glove structure of claim 2, wherein the energy-generating material comprises a positive electromagnetic energy-generating material.

20. The energy-enhanced health-promoting glove structure of claim 3, wherein the energy-generating material comprises a positive electromagnetic energy-generating material.

21. The energy-enhanced health-promoting glove structure of claim 5, wherein the energy-generating material comprises a positive electromagnetic energy-generating material.

22. The energy-enhanced health-promoting glove structure of claim 1, wherein the energy-generating material comprises a mineral material having energy.

23. The energy-enhanced health-promoting glove structure of claim 2, wherein the energy-generating material comprises a mineral material having energy.

24. The energy-enhanced health-promoting glove structure of claim 3, wherein the energy-generating material comprises a mineral material having energy.

25. The energy-enhanced health-promoting glove structure of claim 5, wherein the energy-generating material comprises a mineral material having energy.
